(19) United States
(12) Patent Application Publication
(10) Pub. No.: US 2021/0174922 A1
(43) Pub. Date: Jun. 10, 2021

(54) MEDICAL CARE SUPPORT APPARATUS

(71) Applicant: Canon Medical Systems Corporation, Otawara-shi (JP)

(72) Inventors: Kazumasa NORO, Shioya (JP); Yusuke KANO, Nasushiobara (JP)

(73) Assignee: Canon Medical Systems Corporation, Otawara-shi (JP)

(21) Appl. No.: 17/109,458

(22) Filed: Dec. 2, 2020

(30) Foreign Application Priority Data
Dec. 4, 2019 (JP) 2019-219843

(51) Int. Cl. G16H 15/00; G16H 50/70; G16H 30/20

(52) U.S. Cl. CPC G16H 15/00; G16H 40/20; G16H 30/20; G16H 50/70

(57) ABSTRACT

According to one embodiment, a medical care support apparatus includes processing circuitry. The processing circuitry acquires first grounds information including grounds for a first means to arrive at a prediction of a probability at which an event relating to a patient may occur, acquires second grounds information including grounds for a second means to arrive at a prediction relating to the event, calculates, based on the first grounds information and the second grounds information, a plurality of matching rates indicating a degree to which a plurality of items included in the first grounds information and a plurality of items included in the second grounds information match, and displays an inference result including the plurality of matching rates.

### CROSS-REFERENCE TO RELATED APPLICATIONS

This application is based upon and claims the benefit of priority from Japanese Patent Application No. 2019-219843, filed Dec. 4, 2019, the entire contents of which are incorporated herein by reference.

### FIELD

Embodiments described herein relate generally to a medical care support apparatus.

### BACKGROUND

When a doctor performs medical practice on a patient through treatment and tests, the doctor grasps the condition of the patient based on his/her medical knowledge or past experiences, and decides on the treatment and tests to be performed next.

In recent years, in order to realize a higher quality of treatment, a technique for analyzing enormous volumes of medical care information by an inference system, etc., and presenting information (for example, a side effect occurrence probability after treatment) that is useful to a doctor's decision-making has existed. Furthermore, there is a technique for presenting grounds for inferences of the information inferred by the inference system (inference grounds of the inference system).

However, since these techniques display only the inference grounds of the inference system, it has been difficult to compare these inference grounds with inference grounds of the doctor. Furthermore, since these techniques do not consider the doctor's own inference grounds, the doctor's own inference grounds when making decisions may become obscured.

### BRIEF DESCRIPTION OF THE DRAWINGS

FIG. 1 shows a configuration example of a medical information system in which a medical care support apparatus according to a first embodiment is included.

FIG. 2 shows a configuration example of the medical care support apparatus of FIG. 1.

FIG. 3 is a flowchart exemplifying processing that is executed in processing circuitry of FIG. 2.

FIG. 4 shows a display example of patient list information according to the first embodiment.

FIG. 5 is a flowchart exemplifying medical care decision support processing that is executed in the flowchart of FIG. 3.

FIG. 6 shows a display example of a medical care screen and a selection box according to the first embodiment.

FIG. 7 is a table showing inference information of a user according to the first embodiment.

FIG. 8 is a table showing inference information of a system according to the first embodiment.

FIG. 9 is a table showing a hierarchical corresponding relationship of medical care information according to the first embodiment.

FIG. 10 is a table showing a combination of the corresponding relationship and a correspondence rate according to the first embodiment.

FIG. 11 is a table showing a combination of a matching item and a correspondence rate in inference grounds of the user and the system according to the first embodiment.

FIG. 12 is a table showing a combination of the correspondence rate, a difference in importance level, and a matching rate in each item of the inference grounds of the user and the system according to the first embodiment.

FIG. 13 shows a display example of an inference result according to the first embodiment.

FIG. 14 shows a display example of confirmation information according to the first embodiment.

FIG. 15 shows a display example of warning information according to the first embodiment.

FIG. 16 shows a display example of a medical care screen in which a predetermined item is highlighted according to the first embodiment.

FIG. 17 is a flowchart exemplifying medical care decision support processing according to an application example of the first embodiment.

FIG. 18 shows a display example of confirmation information according to the application example of the first embodiment.

FIG. 19 is a flowchart exemplifying medical care decision support processing according to a second embodiment.

FIG. 20 is a table showing inference information of system A according to the second embodiment.

FIG. 21 is a table showing inference information of system B according to the second embodiment.

FIG. 22 is a table showing a combination of a matching item and a correspondence rate in inference grounds of system A and system B according to the second embodiment.

FIG. 23 shows a combination of the correspondence rate, a difference in importance level, and a matching rate in each item of the inference grounds of system A and system B according to the second embodiment.

FIG. 24 shows a display example of an inference result according to the second embodiment.

FIG. 25 shows a display example of confirmation information according to the second embodiment.

FIG. 26 shows a display example of warning information according to an application example of the second embodiment.

FIG. 27 shows a display example of an inference result according to a third embodiment.

FIG. 28 shows a display example of warning information according to the third embodiment.

### DETAILED DESCRIPTION

In general, according to one embodiment, a medical care support apparatus includes processing circuitry. The processing circuitry acquires first grounds information including grounds for a first means to arrive at a prediction of a probability at which an event relating to a patient may occur, acquires second grounds information including grounds for a second means to arrive at a prediction relating to the event, the second means being different from the first means, calculates, based on the first grounds information and the second grounds information, a plurality of matching rates indicating a degree to which a plurality of items included in the first grounds information and a plurality of items included in the second grounds information match, and displays an inference result including the plurality of matching rates.

Embodiments of the medical care support apparatus will be described in detail below, with reference to the drawings.

### First Embodiment

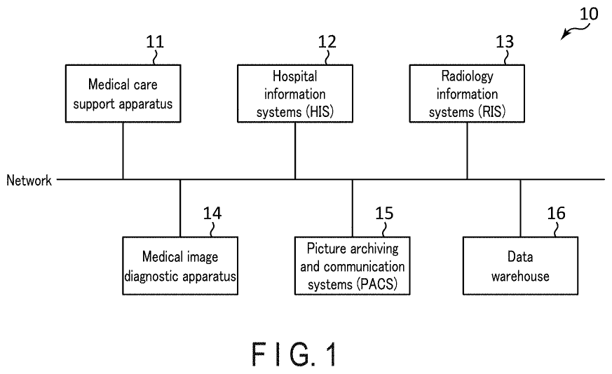

FIG. 1 shows a configuration example of a medical information system in which a medical care support apparatus according to a first embodiment is included. As shown in FIG. 1, a medical information system 10 includes a medical care support apparatus 11, a hospital information systems (HIS) 12, a radiology information systems (RIS) 13, a medical image diagnostic apparatus 14, a picture archiving and communication systems (PACS) 15, and a data warehouse (DWH) 16. The medical information system 10, the medical care support apparatus 11, the HIS 12, the RIS 13, the medical image diagnostic apparatus 14, the PACS 15, and the DWH 16 are connected to each other via a network (in-hospital network) such as a local area network (LAN) in a communicatory manner.

The medical care support apparatus 11 is, for example, an apparatus that is capable of observing a plurality of pieces of medical information integratedly. For example, an integrated viewer is implemented on the medical care support apparatus 11. An integrated viewer is an application that integratedly presents a plurality of pieces of medical information to a user. A medical care screen to be displayed on the integrated viewer is, for example, a screen that is displayed for an operator (user), such as a doctor performing medical practice, to integratedly observe medical care information of a specific patient to perform diagnosis and treatment on the patient. The integrated viewer may adopt any form of implementation, such as a web application, a fat client application, or a thin client application. Hereinafter, the “medical care screen” will be considered as “a medical care screen displayed on an integrated viewer” and described. A specific configuration of the medical care support apparatus 11 will be described later.

The HIS 12 includes, for example, an electronic medical record system for managing information relating to an electronic medical record. The information relating to an electronic medical record includes, for example, patient information and medical care information.

The patient information is information unique to a patient, including, for example, a patient ID, a patient name, gender, birth date, and age. The medical care information is information relating to a patient's physical condition, symptoms, and treatment, etc. obtained by medical professionals in the process of performing medical care. The medical care information includes, for example, image information, test history information, electrocardiogram information, vital sign information, medication history information, report information, information described on a medical record, and nursing record information.

The image information is, for example, information indicating the location of a medical image acquired by imaging a patient, etc. The image information includes, for example, information indicating the location of a medical image file, to be described later, generated by the medical image diagnostic apparatus 14 as a result of performing a test.

The test history information is, for example, information indicating a history of a test result acquired as a result of performing a specimen test and a bacteria test on a patient.

The electrocardiogram information is, for example, information relating to an electrocardiographic complex measured from a patient.

The vital sign information is, for example, basic information relating to the life of a patient. The vital sign information includes, for example, pulse rates, respiratory rates, oxygen concentration, body temperature, blood pressure, and consciousness levels. The medication history information is, for example, information indicating a history of the dosage of medicine administered to a patient.

The report information is, for example, information summarizing the state and disease of a patient obtained by a radiologist of a radiology department reading a medical image such as an X-ray image, a CT image, an MRI image, and an ultrasound image upon receiving a test request from a medical care doctor of a medical care department. The report information includes, for example, image reading report information indicating an image reading report prepared by a radiologist with reference to a medical image file stored in a PACS 15. It should be noted that, in general, since the report information is stored in the PACS 15, the electronic medical record system can display the report information by reading out the report information from the PACS 15.

The information described on a medical record is, for example, information input to the electronic medical record by a medical care doctor, etc. The information described on a medical record includes, for example, a medical care record during hospitalization, a medical history of a patient, and a prescription history of a medicine.

The nursing record information is, for example, information input to the electronic medical record by a nurse, etc. The nursing record information includes a nursing record, etc. during hospitalization.

Furthermore, the information relating to an electronic medical record includes, for example, test implementing information. The test implementing information is generated by the medical image diagnostic apparatus 14 that implemented the test in accordance with test order information. The test implementing information is information indicating the test implemented by the medical image diagnostic apparatus 14. The test implementing information includes an order number, a test unique ID (UID), a patient ID, a modality type, an imaging portion, and an imaging condition, etc.

The test UID is an identifier that can uniquely specify a test. The modality type indicates a modality used for the imaging. The modality type includes, for example, an “X-ray computed tomography apparatus”, an “X-ray diagnostic apparatus”, a “magnetic resonance imaging apparatus”, and an “ultrasound diagnostic apparatus”. The imaging portion corresponds to a test portion included in the test order information. The imaging portion includes, for example, an abdomen, a brain, and a chest. The imaging condition includes postures, imaging directions, and whether to use a contrast agent, etc.

Furthermore, the HIS 12 includes, for example, an order system that manages appointment information and order information, etc. It should be noted that the HIS 12 may also be configured to include the order system in the electronic medical record system.

The appointment information includes, for example, information relating to a consultation appointment and a test appointment. Information relating to the consultation appointment includes, for example, a consultation date, a consultation time, a reception number, a doctor zequest, and a department request. Information relating to the test appointment includes, for example, a test date, a test time, and a reception number.

The order information is, for example, information on the order requested by a medical care doctor, etc. Specifically, the order information includes, for example, an image test, a specimen test, a physiological test, a prescription, and a medication.

In a case where the order information is test order information requesting the image test, the test order information includes, for example, an order number capable of identifying the test, a patient ID, a test type, a test portion, and request source information. The order number is a number that is issued when the test order information is input, and, for example, is for uniquely specifying the test order information in one hospital. The test type includes an X-ray test, a computed tomography (CT) test, a magnetic resonance (MR) test, and a radio isotope (RI) test, etc. The test portion includes, for example, an abdomen, a brain, and a chest. The request source information includes, for example, the name of a medical care department and the name of a doctor in charge. It should be noted that the order information is associated with information relating to the test appointment.

The RIS 13 is a system for managing test appointment information relating to radiograph test affairs. For example, the RIS 13 adds various kinds of setting information to the test order information input by the medical care doctor in the order system included in the HIS 12, accumulates it, and manages the accumulated information as the test appointment information. It should be noted that the RIS 13 may add various kinds of setting information to the test order information by using an irradiation record on which various kinds of setting information set in the medical image diagnostic apparatus 14 in the past tests are recorded. The RIS 13 transmits the test order to the medical image diagnostic apparatus 14 in accordance with the test appointment information. Furthermore, as a result of performing the test, the RIS 13 transmits the test implementing information generated by the medical image diagnostic apparatus 14 to the electronic medical record system included in the HIS 12.

The medical image diagnostic apparatus 14 is an apparatus for implementing the test by imaging a patient, etc. The medical image diagnostic apparatus 14 includes, for example, an X-ray computed tomography apparatus, an X-ray diagnostic apparatus, a magnetic resonance imaging apparatus, a nuclear medicine diagnostic apparatus, and an ultrasound diagnostic apparatus. For example, the medical image diagnostic apparatus 14 implements the test based on the test appointment information transmitted from the RIS 13. The medical image diagnostic apparatus 14 generates the test implementing information and transmits it to the RIS 13.

The medical image diagnostic apparatus 14 also generates medical image data by implementing the test. The medical image data is, for example, X-ray CT image data, X-ray image data, MRI image data, nuclear medicine image data, and ultrasound image data. The medical image diagnostic apparatus 14 generates the medical image file by converting the generated medical image data into, for example, a format in conformity with a digital imaging and communication in medicine (DICOM) standard. The medical image file is, for example, a file in a format in conformity with the DICOM standard. The medical image diagnostic apparatus 14 transmits the generated medical image file to the PACS 15.

The PACS 15 is a system for managing various medical image files. For example, the PACS 15 stores the medical image file transmitted from the medical image diagnostic apparatus 14. It should be noted that the PACS 15 may store report information attached to the medical image file, or report information with respect to tests relating to a plurality of medical image files.

The DWH 16 is a database system that collectively accumulates information generated at, for example, medical care-related institutions, known as medical care big data. The DWH 16 may be realized by, for example, a general server apparatus. The DWH 16 includes, for example, processing circuitry, a memory, and a communication interface. The processing circuitry, the memory, and the communication interface are, for example, connected to each other via a bus in a communicatory manner.

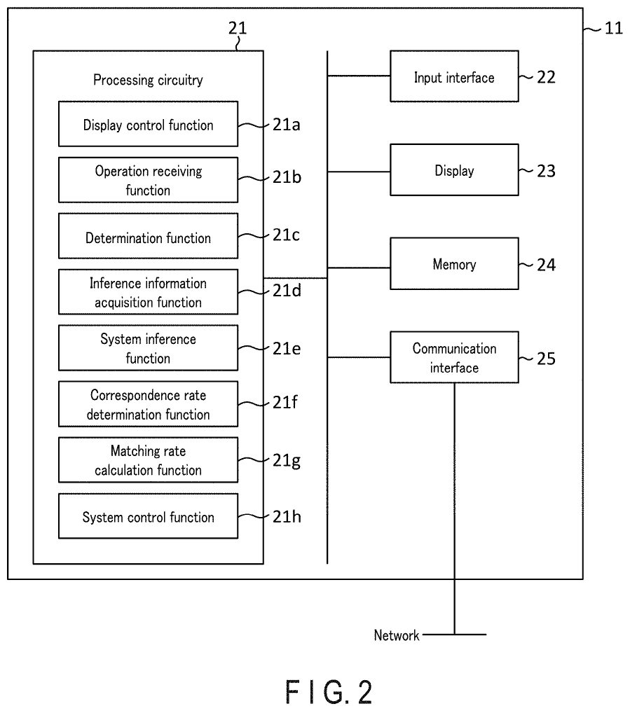

FIG. 2 shows a configuration example of the medical care support apparatus of FIG. 1. As shown in FIG. 2, the medical care support apparatus 11 includes processing circuitry 21, an input interface 22, a display 23, a memory 24, and a communication interface 25. The processing circuitry 21, the input interface 22, the display 23, the memory 24, and the communication interface 25 are, for example, connected to each other via a bus in a communicatory manner.

The processing circuitry 21 includes, as hardware resources, a processor or a memory such as a read-only memory (ROM) and a RAM (not shown) to control the medical care support apparatus 11. The processing circuitry 21 includes a display control function 21a, an operation receiving function 21b, a determination function 21c, an inference information acquisition function 21d, a system inference function 21e, a correspondence rate determination function 21f, a matching rate calculation function 21g, and a system control function 21h. Various functions performed by the display control function 21a, the operation receiving function 21b, the determination function 21c, the inference information acquisition function 21d, the system inference function 21e, the correspondence rate determination function 21f, the matching rate calculation function 21g, and the system control function 21h are stored in the memory in the form of a program executable by a computer. The processing circuitry 21 is a processor for reading out a program corresponding to these functions from the memory and executing it to realize the function corresponding to each program. In other words, the processing circuitry 21 which has read out each program will be able to activate a plurality of functions shown in the processing circuitry 21 of FIG. 2.

FIG. 2 explains a case in which the various functions are realized in a single processing circuitry 21; however, various functions may be realized by a plurality of independent processors executing a program. In other words, the various functions above may be configured as a program, and a single processing circuitry may execute each program, or a specific function may be implemented in dedicated independent program execution circuitry.

The term “processor” used in the above explanation means, for example, a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)).

The processor in the processing circuitry 21 reads out programs stored in the memory 24 and executes them to realize the various functions. The programs may be incorporated directly into circuits of the processor, instead of being stored in the memory 24. In this case, the processor realizes the functions by reading out and executing the programs incorporated into the circuits.

The display control function 21a is a function of displaying display information such as a medical care screen, patient list information, an inference result, confirmation information, and warning information on the display 23. By the display control function 21a, the processing circuitry 21 displays the display information on the display 23. The patient list information, the inference result, the confirmation information, and the warning information will be explained later.

The operation receiving function 21b is a function of receiving a user's operation via the input interface 22 on the medical care screen. The processing circuitry 21 receives the user's operation by the operation receiving function 21b. The user's operation will be explained later.

The determination function 21c is a function of performing various determinations in medical care screen display processing and medical care decision support processing explained later. By the determination function 21c, the processing circuitry 21 performs determination processing using a threshold value and determination processing relating to the user's operation.

The inference information acquisition function 21d is a function of acquiring inference information of a user. By the inference information acquisition function 21d, the processing circuitry 21 acquires the inference information of a user. Inference information is information on events of an inference target (events relating to a patient) predicted by a certain means, and includes the grounds for the certain means to arrive at the prediction. The certain means includes, for example, means of inference by a user, and means of inference by a system, and not a user. Inference information of a user is information on events relating to a patient predicted by a user, and includes the grounds for the user to arrive at the prediction. The inference information may be referred to as grounds information. A specific example of the grounds information will be explained later.

Specifically, the processing circuitry 21 acquires information on inference grounds of the user (second grounds information) relating to events of the inference target (events relating to a patient) by an input from a user. In other words, the processing circuitry 21 acquires the second grounds information which represents events relating to the patient predicted by the user, and includes the grounds for the user to arrive at the prediction. The inference target will be explained later.

The system inference function 21e is a function of generating a probability at which the inference target may occur (prediction probability) and executing an inference by the system. By the system inference function 21e, the processing circuitry 21 generates the prediction probability and inference information of the system. The inference information of the system is information on an occurrence probability of an event relating to the patient being predicted by the system, and includes grounds for the system to arrive at the prediction.

Specifically, the processing circuitry 21 uses a model trained based on machine learning relating to the inference target, and outputs a prediction probability at which the inference target may occur to a target patient and inference grounds contributing to the calculation of the prediction probability. In other words, the processing circuitry 21 predicts the probability at which the event relating to the patient may occur, and acquires information on the inference grounds for the system (first grounds information) to arrive at the prediction. The inference grounds of the system may be extracted not only from data relating to the target patient, but also from, for example, data relating to similar cases, clinical guidelines, and medical papers.

The correspondence rate determination function 21f is a function of determining correspondence rates of a plurality of inference grounds. By the correspondence rate determination function 21f, the processing circuitry 21 determines the correspondence rate between the information on the user's inference grounds (second grounds information) and the information on the system's inference grounds (first grounds information), or the correspondence rate of inference grounds among a plurality of systems. In other words, the processing circuitry 21 determines a plurality of correspondence rates indicating the corresponding relationship of a plurality of items included in the first grounds information and a plurality of items included in the second grounds information, respectively. The correspondence rate will be explained later.

The matching rate calculation function 21g is a function of determining matching rates of a plurality of inference grounds. By the matching rate calculation function 21g, the processing circuitry 21 calculates the matching rate between the user's inference grounds and the system's inference grounds, or the matching rate of inference grounds of a plurality of systems. Specifically, the processing circuitry 21 calculates a plurality of matches indicating the degree to which a plurality of items included in the first grounds information and a plurality of items included in the second grounds information match based on the first grounds information provided by the system and the second grounds information provided by the user. More specifically, the processing circuitry 21 calculates a plurality of matching rates based on a plurality of first importance levels associated with each of a plurality of items included in the first grounds information and a plurality of second importance levels associated with each of a plurality of items included in the second inference grounds. The matching rate will be explained later.

In a case where a plurality of correspondence rates are determined, by the matching rate calculation function 21g, the processing circuitry 21 calculates a plurality of matching rates based on a plurality of first importance levels, a plurality of second importance levels, and a plurality of correspondence rates. Specifically, the processing circuitry 21 calculates the difference in importance level for each item included in the first grounds information and the second grounds information based on the difference between the first importance level and the second importance level, and calculates the matching rate based on the difference between the correspondence rate and the importance level. Alternatively, the processing circuitry 21 calculates the difference in importance level for each item above based on a difference in rank order between a first rank order corresponding to the first importance level and a second rank order corresponding to the second importance level, and calculates the matching rate based on the difference between the correspondence rate and the importance level.

As for the specific calculation on the matching rate, the processing circuitry 21 calculates the matching rate for each of the items above based on a ratio between the correspondence rate and the difference in importance level. Alternatively, in a case where the difference in importance level is normalized in a predetermined numerical value range, the processing circuitry 21 calculates the matching rate for each of the items above by multiplying the correspondence rate by a value obtained by subtracting the difference in importance level from a maximum value of the predetermined numerical value range.

The system control function 21h is a function of controlling basic operations of the medical care support apparatus 11, such as input/output and communications. When the system control function 21h is executed, the processing circuitry 21 receives various requests via the input interface 22. The processing circuitry 21 executes various functions in accordance with the various requests that are received.

The display control function 21a, the operation receiving function 21b, the determination function 21c, the inference information acquisition function 21d, the system inference function 21e, the correspondence rate determination function 21f, the matching rate calculation function 21g, and the system control function 21h may be incorporated into the processing circuitry 21 as a control program, or a dedicated hardware circuit capable of implementing each function above may be incorporated into the processing circuitry 21 itself.

The input interface 22 is realized by, for example, a mouse, a keyboard, or a touch panel to which an instruction is input by touching an operation surface. The input interface 22 receives, for example, an operation conducted by a user. The user's operation corresponds to, for example, a moving operation, a click operation, and a drag-and-drop operation of a mouse pointer. The input interface 22 converts the operation conducted by the user into an electrical signal, and outputs the electrical signal to the processing circuitry 21.

The display 23 displays various kinds of information for the user to conduct various tasks. For example, the display 23 is controlled by the processing circuitry 21 to display the medical care screen. As the display 23, for example, a CRT display, a liquid crystal display, an organic EL display, an LED display, a plasma display, and any other display known in the relevant technical field may be used as appropriate.

The memory 24 stores various kinds of information. As the memory 24, for example, a hard disk drive (HDD), a solid state drive (SSD), and an integrated circuit storage device may be used as appropriate. The memory 24 may also be a driving device, etc., that reads and writes various kinds of information to and from portable storage media, such as a CD-ROM drive, a DVD drive, and a flash memory.

The communication interface 25 performs data communication with the HIS 12, the RIS 13, the medical image diagnostic apparatus 14, the PACS 15, and the DWH 16 connected thereto via the in-hospital network. The communication standard for the HIS 12, the RIS 13, the medical image diagnostic apparatus 14, the PACS 15, and the DWH 16 may be any standard, including examples such as HL7, DICOM, or both.

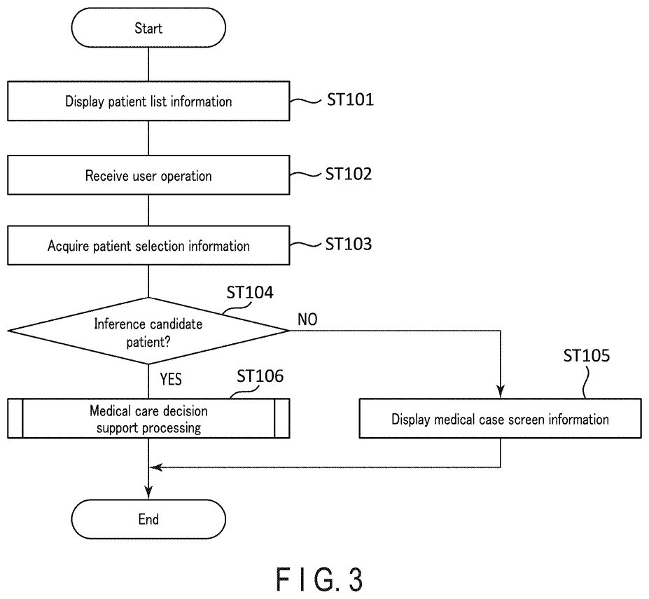

FIG. 3 is a flowchart exemplifying the medical care screen display processing that is executed in the processing circuitry of FIG. 2. For example, depending on whether or not the patient is the inference candidate, the medical care screen display processing further executes medical care decision support processing explained later or displays a usual medical care screen. The processing of FIG. 3 is started by, for example, a user, etc. executing an operation of displaying a patient list.

(Step ST101)

Once an operation is performed by the user, the processing circuitry 21 executes the display control function 21a. By the display control function 21a, the processing circuitry 21 displays the patient list information on the display 23. The patient list information is, for example, information on a list of patients the user is in charge of, information on a list of patients suffering from the same disease, and information on a list of patients who are to receive the same treatment.

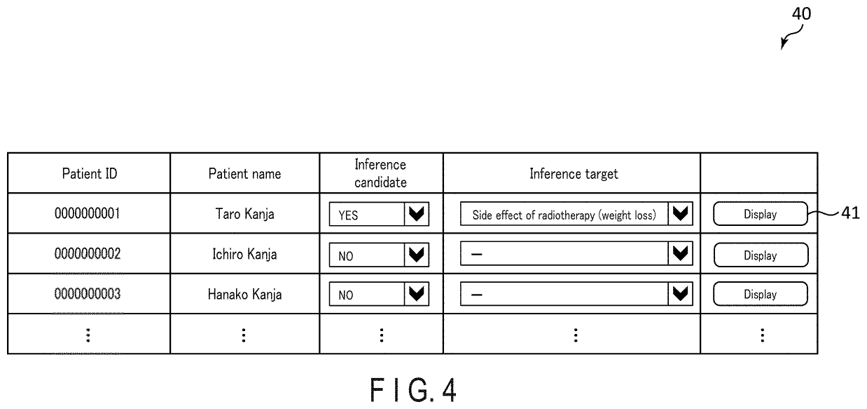

FIG. 4 shows a display example of the patient list information according to the first embodiment. In patient list information 40 shown in FIG. 4, a patient ID and name, a drop-down list for selecting whether or not the patient is an inference candidate, a drop-down list for selecting the inference target, and a display button are associated. The inference target is a generic term for events that require the user's decision on the patient's medical care. The inference target includes, for example, “side effect of radiotherapy (weight loss)”, “use of anticancer agent”, and “radiotherapy”.

(Step ST102)

After the patient list information is displayed, the processing circuitry 21 executes the operation receiving function 21b. The processing circuitry 21 receives the user's operation by the operation receiving function 21b. In the present embodiment, for example, the user is assumed to select “YES” for the inference candidate and “side effect of radiotherapy (weight loss)” for the inference target with respect to a patient ID “0000000001” and a patient name “Taro Kanja”, and press the display button 41.

(Step ST103)

After receiving the operation of the display button 41 being pressed by the user, the processing circuitry 21 acquires patient selection information. The patient selection information is, for example, information associating the patient ID “0000000001”, the patient name “Taro Kanja”, the inference candidate “YES”, and the inference target “side effect of radiotherapy (weight loss)”.

(Step ST104)

After acquiring the patient selection information, the processing circuitry 21 executes the determination function 21c. By the determination function 21c, the processing circuitry 21 determines whether or not the patient selected by the user is an inference candidate patient. In a case where the patient selected by the user is not an inference candidate patient (that is, in a case where the inference candidate is “NO”), the processing proceeds to step ST105, and, in a case where the patient selected by the user is an inference candidate patient (that is, in a case where the inference candidate is “YES”), the processing proceeds to step ST106.

(Step ST105)

By the display control function 21a, the processing circuitry 21 displays the medical care screen on the display 23. After the medical care screen is displayed, the medical care screen display processing is ended. Detailed explanations of the medical care screen will be given later.

(Step ST106)

The processing circuitry 21 executes the medical care decision support processing. By executing the medical care decision support processing, the medical care screen display processing is ended.

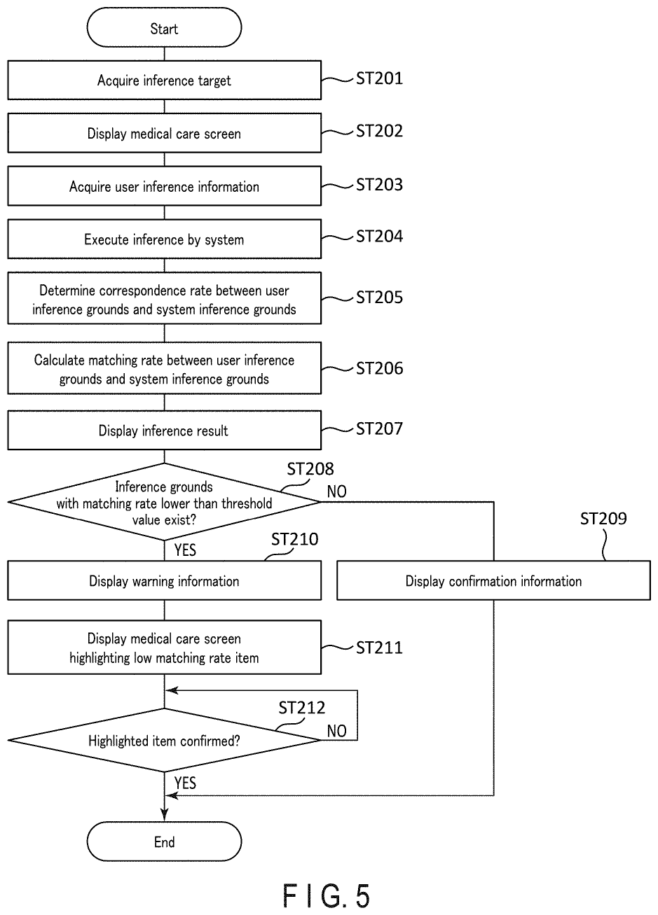

FIG. 5 is a flowchart exemplifying the medical care decision support processing that is executed in the flowchart of FIG. 3.

(Step ST201)

When the medical care decision support processing is executed, the processing circuitry 21 acquires information on the inference target selected by the user. For example, the processing circuitry 21 acquires information on the inference target “side effect of radiotherapy (weight loss)”. The information on the inference target that is acquired here determines the target to be inferred by the system explained later.

(Step ST202)

After acquiring the information on the inference target, by the display control function 21a, the processing circuitry 21 displays the medical care screen on the display 23. The displayed medical care screen is referred to when the user predicts the inference grounds of the inference target. This medical care screen may include a selection box for assisting the user to predict a side effect.

The medical care screen includes, for example, a region indicating a timeline (hereinafter referred to as a timeline region) and a region indicating medical care information (hereinafter referred to as a medical care information region).

The timeline region is, for example, a region indicating a plurality of pieces of medical care information for a specific patient in time sequence, in the order of date and time. Examples of the medical care information include “Event” indicating information on a treatment plan (Planning treatment plan), a feedback session (Conference), a planned period (Treatment), etc. of a specific patient, “Labs” (specimen test) corresponding to test history information of an electronic medical record, “Imaging” (image test) corresponding to image information of the electronic medical record, “Imaging Measurements” (image test measurement) corresponding to a measurement result of the image test, “Clinical Notes” (medical record description) and “Nursing Notes” (nursing record), etc. Each piece of individual medical care information above is arranged in the timeline region in the form of an individual icon.

The medical care information region includes, for example, one or more regions capable of displaying individual medical care information. Specifically, for example, at least one of image information in the image test (Imaging), medicine administration information (Medications), vital sign information (Vitals), or specimen test information (Labs) may be displayed in the medical care information region. For example, in a rectangular medical care information region, the image information may be displayed in a left column region, the medicine administration information may be displayed in an upper region of a center column, the vital sign information may be displayed in a lower region of the center column, and the specimen test information may be displayed in a right column region. In the medical care information region, the arrangement of the medical care information and the type of the medical care information may be changed as appropriate by the user performing the operation.

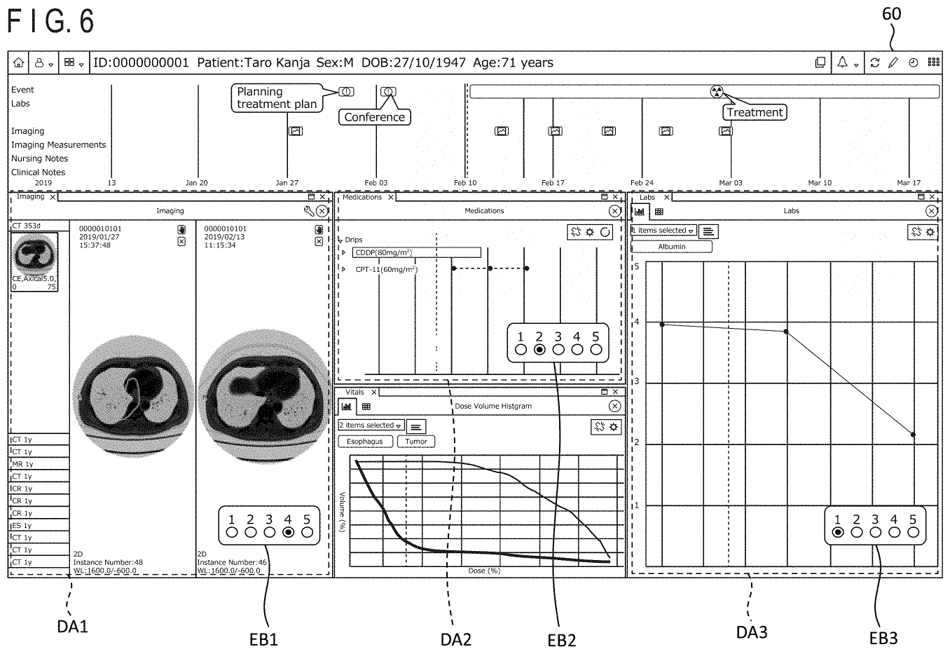

FIG. 6 shows a display example of the medical care screen and the selection box according to the first embodiment. A medical care screen 60 shown in FIG. 6 includes the timeline region, the medical care information region, and a plurality of selection boxes. The medical care information region includes an image information region DA1, a medicine administration information region DA2, a vital sign information region, and a specimen test information region DA3. Furthermore, a selection box EB1, a selection box EB2, and a selection box EB3 are displayed respectively in the image information region DA1, the medicine administration information region DA2, and the specimen test information region DA3 in an overlapping manner.

(Step ST203)

After the medical care screen is displayed, the processing circuitry 21 executes the inference information acquisition function 21d. By the inference information acquisition function 21d, the processing circuitry 21 acquires inference information of the user (user inference information). The user inference information includes, for example, at least the inference grounds presenting the grounds for the inference target to be selected by the user. The inference grounds include, for example, “CT image_left lung” displayed in the image information region DA1, “Injection: CPT-11” displayed in the medicine administration information region DA2, and “Blood test: Albumin” displayed in the specimen test information region DA3 in FIG. 6.

The user inference information may also include an importance level of the inference grounds selected by the user. For example, the importance level may be determined by the user selecting a numeric value in the selection box displayed on the medical care screen. In the present embodiment, the numeric value indicating an importance level that can be selected by the user is presented in five stages from “1” to “5”, in which the importance level becomes higher as the numeric number becomes larger.

For example, in the medical care screen of FIG. 6, the user selects “4” in the selection box EB1, selects “2” in the selection box EB2, and selects “1” in the selection box EB3. It should be noted that the number of selection boxes to be displayed is not limited. Therefore, for example, the selection box may also be displayed in a vital information region in an overlapping manner. Furthermore, in a case where the user displays new medical care information (for example, clinical records (Clinical notes)), a selection box is also displayed in the region of the newly displayed medical care information.

The user inference information may also include a rank order relating to the importance level of the inference grounds. For example, the processing circuitry 21 determines the rank order based on the value of the importance level. Specifically, the processing circuitry 21 determines the rank order in descending order from high numeric values of the importance level.

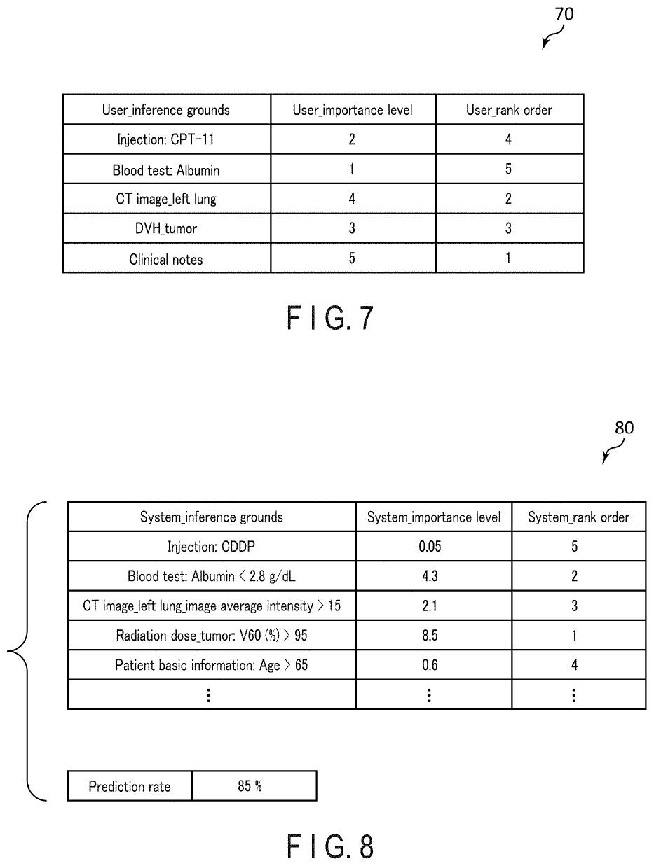

FIG. 7 is a table showing the user inference information according to the first embodiment. In a table 70 shown in FIG. 7, the inference grounds selected by the user (user_inference grounds), the importance level of the inference grounds (user_importance level), and the rank order of the importance level (user_rank order) are associated. As an example, when the user selects “4” in the selection box EB1, inference grounds “CT image_left lung” and importance level “4” are associated. In FIG. 2, for example, inference grounds “CT image_left lung”, importance level “4”, and rank order “2” are associated.

(Step ST204)

After acquiring the user inference information, the processing circuitry 21 executes the system inference function 21e. By the system inference function 21e, the processing circuitry 21 executes the inference by the system and generates the inference information of the system (system inference information). The system inference information includes, for example, at least the inference grounds and the importance level. The system inference information may also include a rank order. The system inference information may also include a prediction probability relating to the inference target.

FIG. 8 is a table showing the system inference information according to the first embodiment. In a table 80 shown in FIG. 8, the inference grounds inferred by the system (system_inference grounds), the importance level (system_importance level), and the rank order (system_rank order) are associated. In FIG. 8, for example, inference grounds “CT image_left lung_image average intensity >15”, importance level “2.1”, and rank order “3” are associated.

In FIG. 8, a prediction probability of the inference target “side effect of radiotherapy (weight loss)” provided by the system is also shown. Specifically, based on the inference grounds and the importance level associated in the table 80, a prediction probability “85%” is shown.

(Step ST205)

After executing the inference by the system, the processing circuitry 21 executes the correspondence rate determination function 21f. By the correspondence rate determination function 21f, the processing circuitry 21 determines a correspondence rate between user inference grounds and system inference grounds. The correspondence rate is a numeric value that is determined by a granularity of the medical care information when comparing the inference grounds selected by the user and the inference grounds inferred by the system. For example, the granularity of the medical care information corresponds to a hierarchy that matches when the medical care information is hierarchically classified. For example, in a case were certain inference grounds are classified into three hierarchies such as a large item, an intermediate item, and a small item, if there is a match in the small item, the granularity of the medical care information is fine, and a high value is set for the correspondence rate. On the other hand, if there is a match in the large item, the granularity of the medical care information is coarse, and a low value is set for the correspondence rate.

Specifically, the processing circuitry 21 uses a table in which the medical care information is associated in a hierarchically classified manner to search for a matching item between the user inference grounds and the system inference grounds, and determines the correspondence rate in accordance with the hierarchy of the matching item (that is, the corresponding relationship on which item is a match). Furthermore, when determining the correspondence rate, the processing circuitry 21 may also generate information in which the matching item and the correspondence rate are associated with each other in the user inference grounds and the system inference grounds.

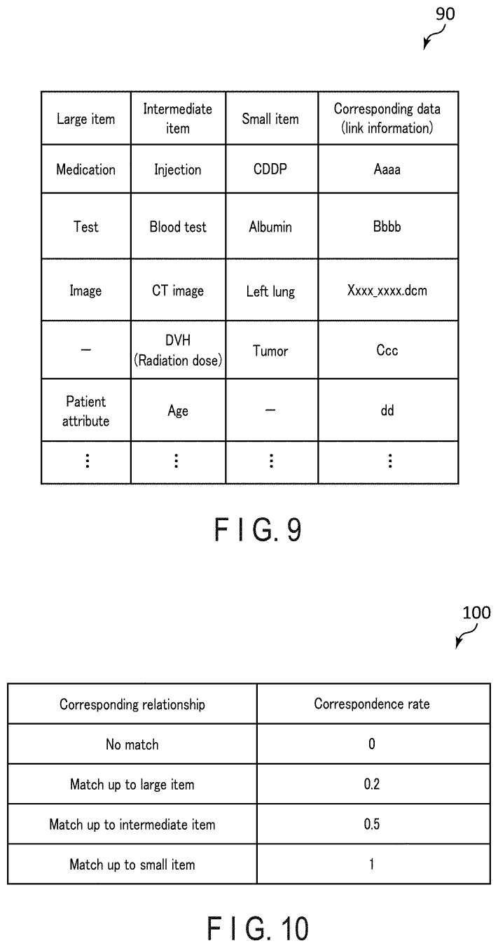

FIG. 9 is a table showing a hierarchical corresponding relationship of the medical care information according to the first embodiment. A table 90 shown in FIG. 9 associates the large item, the intermediate item, the small item, and corresponding data (link information) with respect to the medical care information. In FIG. 9, for example, a large item “image”, an intermediate item “CT image”, a small item “left lung”, and corresponding data (link information) “Xxxx_xxxx.dcm” are associated with each other.

FIG. 10 is a table showing a combination of the corresponding relationship and the correspondence rate according to the first embodiment. A table 100 shown in FIG. 10 associates the corresponding relationship and the correspondence rate with respect to the matching item. In FIG. 10, for example, the corresponding relationship “match up to small item” and the correspondence rate “1” are associated with each other.

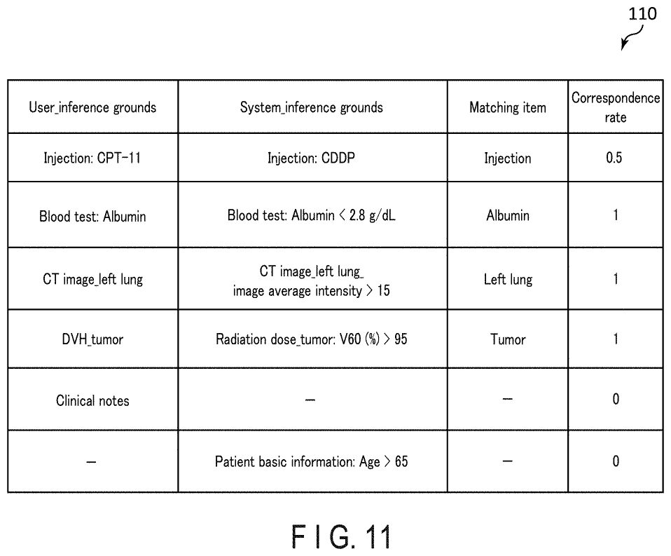

FIG. 11 is a table showing a combination of the matching item and the correspondence rate in the user and the system inference grounds according to the first embodiment. In a table 110 shown in FIG. 11, the user_inference grounds, the system_inference grounds, the matching item, and the correspondence rate are associated with each other. In FIG. 11, for example, the user_inference grounds “CT image_left lung”, the system_inference grounds “CT image_left lung_image average intensity >15”, the matching item “left lung”, and the correspondence rate “1” are associated with each other. Furthermore, for example, since the user_inference grounds “Clinical notes” was not extracted in the system's inference, the matching item “-” (that is, no matching item) and the correspondence rate “0” are shown. In the same manner, since the system_inference grounds “Patient basic information: Age >65” was not selected in the user's inference, the matching item “-” and the correspondence rate “0” are shown.

(Step ST206)

After determining the correspondence rate, the processing circuitry 21 executes the matching rate calculation function 21g. By the matching rate calculation function 21g, the processing circuitry 21 calculates the matching rate between the user inference grounds and the system inference grounds. The matching rate is an indicator indicating the degree to which the user's inference grounds and the system's inference grounds match. Specifically, the matching rate is calculated based on the corresponding relationship between the item in the user inference grounds and the item in the system inference grounds and the importance level of each item.

For example, the processing circuitry 21 calculates the matching rate based on the correspondence rate and the difference in importance level. The difference in importance level is calculated based on the importance level of the user inference grounds and the importance level of the system inference grounds. Furthermore, the difference in importance level may be calculated based on a difference in rank orders between the rank order of the user inference grounds and the rank order of the system inference grounds. It should be noted that the difference in importance level is normalized to stay within a predetermined numerical value range (for example, zero to one).

As an example of calculating the matching rate, the processing circuitry 21 calculates the matching rate for each item based on a ratio between the correspondence rate and the difference in importance level. Specifically, the processing circuitry 21 calculates the matching rate for each item by multiplying the “correspondence rate” by “a value obtained by subtracting the difference in importance level from a maximum value of the predetermined numerical value range”. Furthermore, when calculating the matching rate, the processing circuitry 21 may also generate information in which the correspondence rate, the difference in importance level, and the matching rate are associated with each other in each item of the user and the system inference grounds.

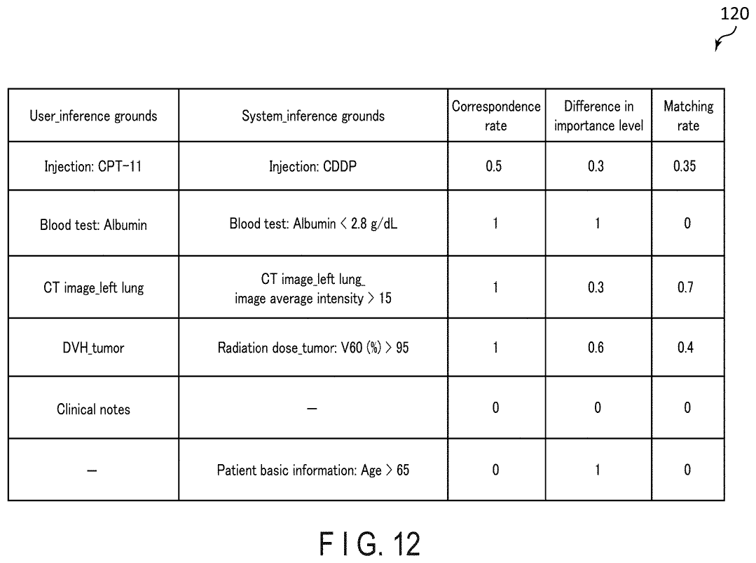

FIG. 12 is a table showing a combination of the correspondence rate, the difference in importance level, and the matching rate in each item of the user and the system inference grounds according to the first embodiment. In a table 120 shown in FIG. 12, the user_inference grounds, the system_inference grounds, the correspondence rate, the difference in importance level, and the matching rate are associated with each other. In FIG. 12, for example, the user inference grounds “CT image_left lung”, the system_inference grounds “CT image_left lung_image average intensity >15”, the correspondence rate “1”, the difference in importance level “0.3”, and the matching rate “0.7” are associated with each other.

(Step ST207)

After calculating the matching rate, by the display control function 21a, the processing circuitry 21 displays an inference result on the display 23. For example, the inference result includes a prediction probability relating to the inference target and a table indicating a user and system inference grounds matching rate. In the table indicating the inference grounds matching rate, for example, the user inference information, the system inference information, and the matching rate thereof are associated with each other. In other words, in the inference result, a plurality of items included in the first grounds information, a plurality of items included in the second grounds information, and a plurality of matching rates are associated with each other, respectively.

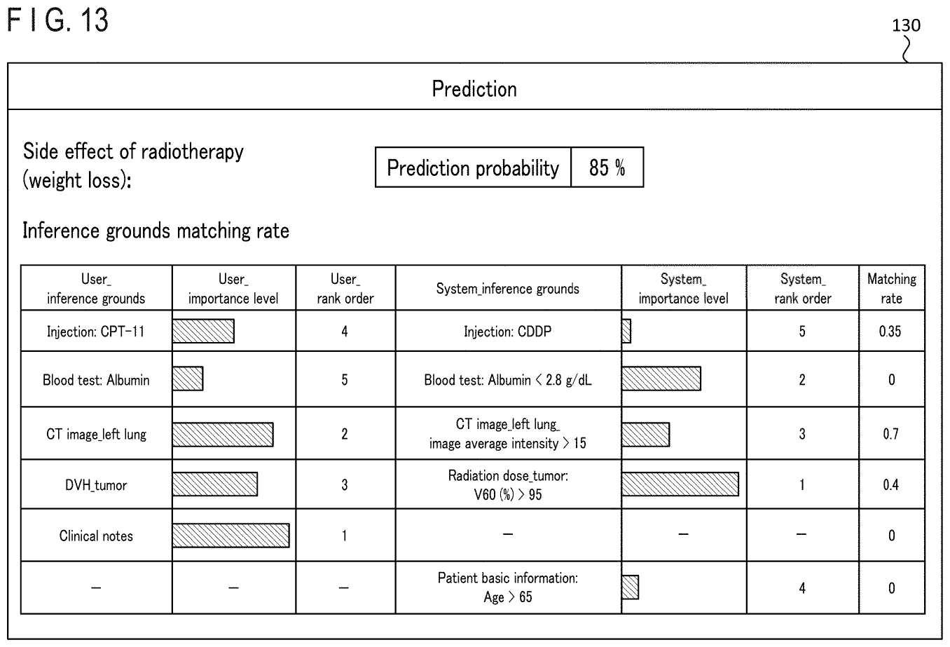

FIG. 13 shows a display example of the inference result according to the first embodiment. In an inference result 130 shown in FIG. 13, a prediction probability relating to the side effect of radiotherapy (weight loss) is shown. Furthermore, in the inference result 130, a table in which the user_inference grounds, the user_importance level, the user_rank order, the system_inference grounds, the system_importance level, the system_rank order, and the matching rate are associated with each other is shown.

Specifically, in the inference result 130, the prediction probability is indicated as “85.”. Furthermore, in the inference result 130, as an example of the inference grounds matching rate, the user inference grounds “CT image_left lung”, a graph corresponding to the user_importance level “4”, the user_rank order “2”, the system_inference grounds “CT image_left lung_image average intensity >15”, a graph corresponding to the system_importance level “2.1”, the system_rank order “3”, and the matching rate “0.7” are shown in an associated manner. Although the user_importance level and the system_importance level are expressed by bar graphs whose widths are each based on respective values, they may also be expressed by actual values.

(Step ST208)

After the inference result is displayed, by the determination function 21c, the processing circuitry 21 determines whether or not there are inference grounds with a matching rate lower than a threshold value in the inference result. In the case where there are no inference grounds with a matching rate lower than the threshold value, the processing proceeds to step ST209, and in the case where there are inference grounds with a matching rate lower than the threshold value, the processing proceeds to step ST210.

(Step ST209)

After it is determined that there are no inference grounds with a matching rate lower than the threshold value, by the display control function 21a, the processing circuitry 21 displays confirmation information indicating that the user and the system inferences are a match on the display 23. After the confirmation information is displayed, the medical care decision support processing is ended.

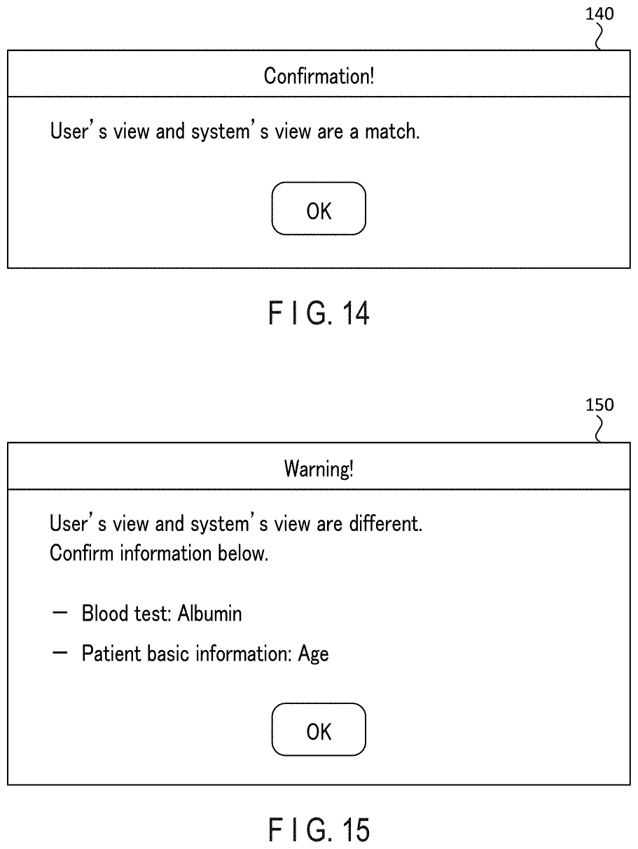

FIG. 14 shows a display example of the confirmation information according to the first embodiment. In confirmation information 140 shown in FIG. 14, for example, a text such as “User's view and system's view are a match” is displayed.

(Step ST210)

After it is determined that there are inference grounds with a matching rate lower than the threshold value, by the display control function 21a, the processing circuitry 21 displays warning information indicating that there is a difference between the user and the system inferences on the display 23.

FIG. 15 shows a display example of the warning information according to the first embodiment. In warning information 150 shown in FIG. 15, for example, texts such as “User's view and system's view are different. Confirm information below.”, “Blood test: Albumin”, and “Patient basic information: Age” are displayed.

As for the item “Blood test: Albumin” displayed on the warning information 150, since there is a wide gap in the importance level between the user and the system, the matching rate is indicated as “0”. Therefore, since there is a possibility that the user may have overlooked the item, the information is included in the warning information 150.

Furthermore, the item “Patient basic information: Age” displayed in the warning information 150 corresponds to the item “Patient basic information age >65” of the system_inference grounds. Since this item was not presented as the inference grounds by the user, the matching rate is indicated as “0”. Therefore, since there is a possibility that the user may have overlooked the item, the item is included in the warning information 150.

(Step ST211)

After the warning information is displayed, by the display control function 21a, the processing circuitry 21 displays the medical care screen on which an item with a low matching rate is highlighted on the display 23. The low matching rate indicates, for example, a value lower than the threshold value that is determined in step ST208. Specifically, for example, the processing circuitry 21 displays an individual medical care information region corresponding to an item with a low matching rate on the medical care screen in a highlighted manner.

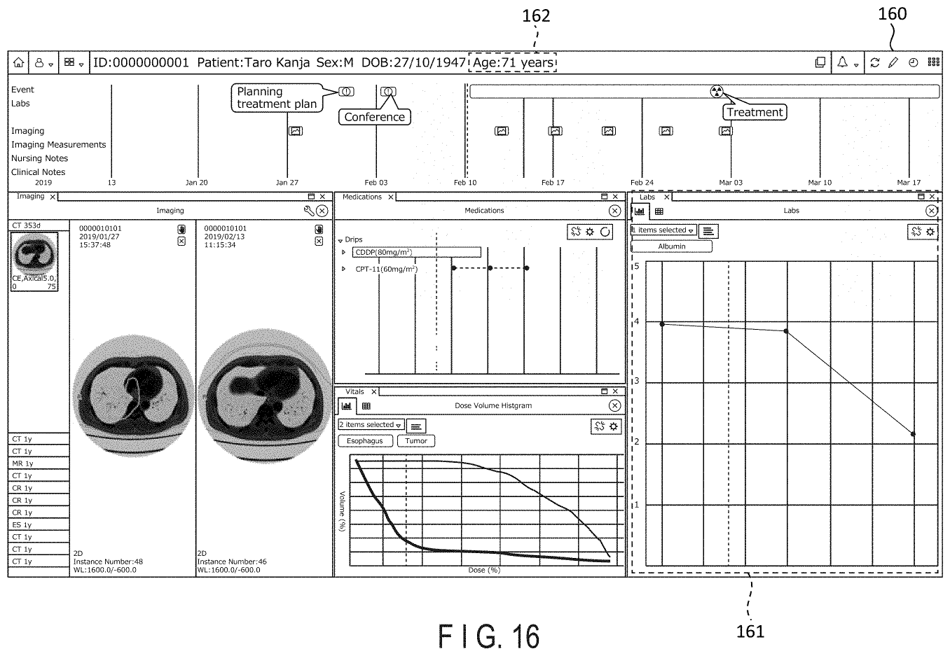

FIG. 16 shows a display example of the medical care screen on which a predetermined item is highlighted according to the first embodiment. In a medical care screen 160 shown in FIG. 16, a specimen test information region 161 corresponding to the item “Blood test: Albumin” and an age information region 162 corresponding to the item “Patient basic information: Age” are highlighted and displayed. By highlighting the items to be confirmed by the user, the user may easily confirm the items. Depending on the items to be highlighted, the processing circuitry 21 may change and display the content of the medical care information region on the medical care screen.

(Step ST212)

After displaying the medical care screen on which the item with a low matching rate is highlighted, by the determination function 21c, the processing circuitry 21 determines whether or not the user has confirmed the highlighted item. The user confirms the item by, for example, clicking the highlighted item. In the case where the user has not confirmed the highlighted item (that is, in the case where the highlighted item remains), the processing is suspended until the user's confirmation is completed. In the case where the user has confirmed the highlighted item, the medical care decision support processing is ended.

As explained above, the medical care support apparatus according to the first embodiment predicts the probability at which the event relating to the patient may occur by the first means, acquires the first grounds information forming the grounds for the first means to arrive at the prediction, predicts the event by the second means which is different from the first means, acquires the second grounds information forming the grounds for the second means to arrive at the prediction, calculates, based on the first grounds information and the second grounds information, a plurality of matching rates indicating the degree to which a plurality of items included in the first grounds information and a plurality of items included in the second grounds information match, and displays the inference result including a plurality of matching rates. In the first embodiment, the second means is, for example, an input from the user. Therefore, since the medical care support apparatus is able to display the matching rate that is calculated after separately acquiring the information on the system inference grounds (first grounds information) and the user inference grounds (second grounds information), both the system and the user inference grounds can be used for the decision without making the inference grounds of the user himself/herself ambiguous. Accordingly, the medical care support apparatus is able to improve the accuracy of the decision.

### Application Example of First Embodiment

In an application example of the first embodiment, processing in a case where there is a deficiency in the user inference information in the medical care decision support processing will be explained. When there is a deficiency in the user inference information, for example, the number of items of the inference grounds given by the user is small, and the items of the inference grounds given by the user hardly match the items of the system inference grounds.

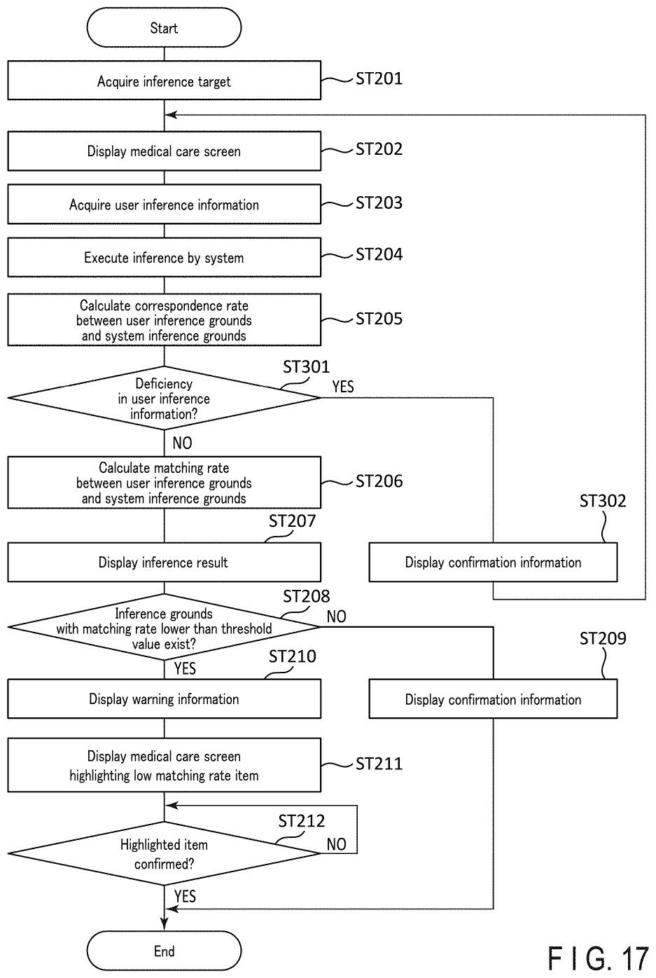

FIG. 17 is a flowchart exemplifying the medical care decision support processing according to the application example of the first embodiment. In the flowchart shown in FIG. 17, a determination step relating to the user inference information is added between step ST205 and step ST206 in the flowchart of FIG. 5.

(Step ST301)

After determining the correspondence rate in step ST205, the processing circuitry 21 executes the determination function 21c. By the determination function 21c, the processing circuitry 21 determines whether or not there is a deficiency in the user inference information. Specifically, among items of the correspondence rate corresponding to the user inference grounds, the processing circuitry 21 determines whether or not the item of the correspondence rate equal to or higher than a threshold value is lower than a predetermined number. In a case where the item of the correspondence rate equal to or higher than the threshold value is lower than the predetermined number, it is considered that there is a deficiency in the user inference information, and the processing proceeds to step ST302. In a case where the item of the correspondence rate equal to or higher than the threshold value is equal to or higher than the predetermined number, it is considered that there is no deficiency in the user inference information, and the processing proceeds to step ST206.

(Step ST302)

After it is determined that there is a deficiency in the user inference information, by the display control function 21a, the processing circuitry 21 displays confirmation information indicating that there is a deficiency in the user inference grounds on the display 23.

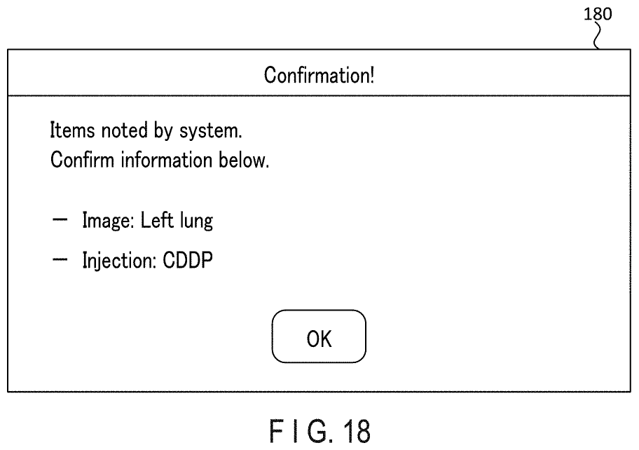

FIG. 18 shows a display example of the confirmation information according to the application example of the first embodiment. In confirmation information 180 shown in FIG. 18, for example, texts such as “Items noted by system. Confirm information below”, “Image: Left lung”, and “Injection: CDDP” are displayed.

After the confirmation information is displayed in step ST302, the processing returns to step ST202.

In the manner explained above, the medical care support apparatus according to the application example of the first embodiment can prevent the user from overlooking the inference.

### Second Embodiment

In the first embodiment, the matter of displaying the inference result obtained by comparing the user inference grounds with the system inference grounds is explained. On the other hand, in a second embodiment, a matter of displaying an inference result obtained by comparing inference grounds between a plurality of systems will be explained. Hereinafter, as a plurality of systems, for example, two systems, such as system A and system B, are assumed. System A and system B are assumed as using different algorithms. Furthermore, the different algorithms include a trained model which has been trained by different training data.

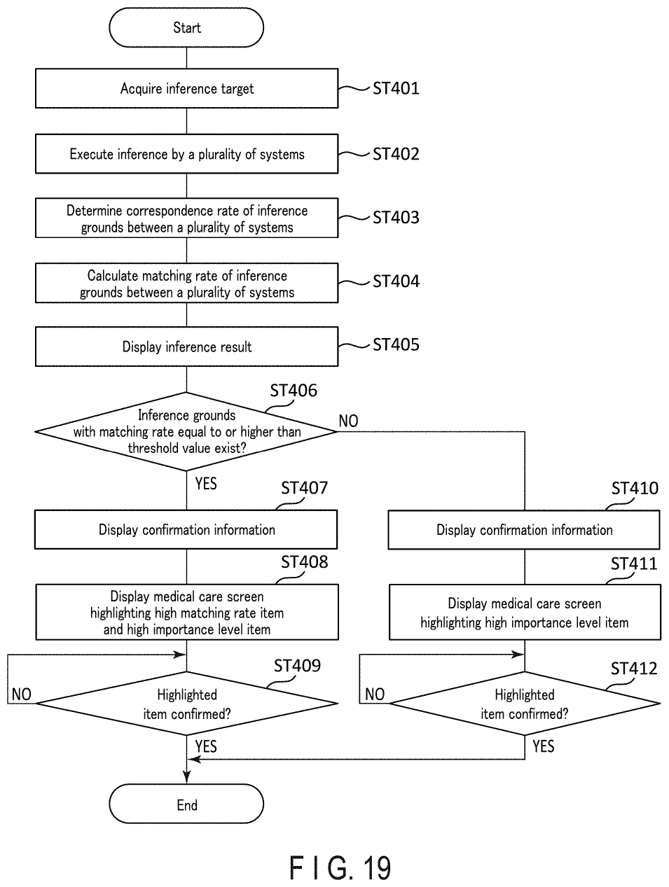

FIG. 19 is a flowchart exemplifying medical care decision support processing according to a second embodiment.

(Step ST401)

When the medical care decision support processing is executed, processing circuitry 21 acquires information on an inference target selected by a user. For example, the processing circuitry 21 acquires information on an inference target “side effect of radiotherapy (weight loss)”. The information on the inference target that is acquired here determines the target to be inferred by a plurality of systems explained later.

(Step ST402)

After acquiring the information on the inference target, the processing circuitry 21 executes a system inference function 21e. By the system inference function 21e, the processing circuitry 21 executes an inference by a plurality of systems and generates a plurality of pieces of system inference information. The plurality of pieces of system inference information include, for example, inference information of system A and inference information of system B.

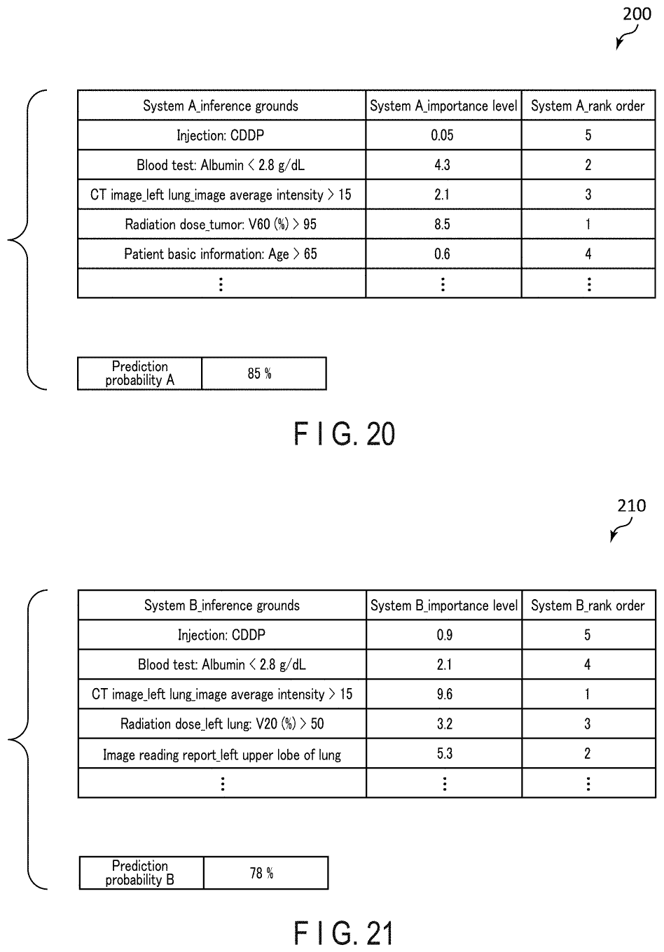

FIG. 20 is a table showing the inference information of system A according to the second embodiment. In a table 200 shown in FIG. 20, inference grounds inferred by system A (system A_inference grounds), an importance level (system A_importance level), and a rank order (system A_rank order) are associated with each other. In FIG. 20, for example, the inference grounds “Blood test: Albmin <2.8 g/dL”, the importance level “4.3”, and the rank order “2” are associated with each other.

In FIG. 20, a prediction probability of an inference target “side effect of radiotherapy (weight loss)” by system A is also shown. Specifically, based on the inference grounds and the importance level associated in the table 200, a prediction probability “85%” is shown.

FIG. 21 is a table showing inference information of system B according to the second embodiment. In a table 210 shown in FIG. 21, inference grounds inferred by system B (system B_inference grounds), an importance level (system B_importance level), and a rank order (system B_rank order) are associated with each other. In FIG. 21, for example, the inference grounds “CT image_left lung_image average intensity >15”, the importance level “9.6”, and the rank order “1” are associated with each other.

In FIG. 21, a prediction probability of an inference target “side effect of radiotherapy (weight loss)” by system B is also shown. Specifically, based on the inference grounds and the importance level associated in the table 210, a prediction probability B “78%” is shown.

(Step ST403)

After executing the inference by the plurality of systems, the processing circuitry 21 executes a correspondence rate determination function 21f. By the correspondence rate determination function 21f, the processing circuitry 21 determines the correspondence rate of the inference grounds between the plurality of systems. Here, the correspondence rate is a numeric value that is determined by a granularity of medical care information when comparing each of the inference grounds inferred by the plurality of systems. Furthermore, when determining the correspondence rate, the processing circuitry 21 may also generate information in which a matching item and the correspondence rate are associated with each other in the inference grounds of system A and system B.

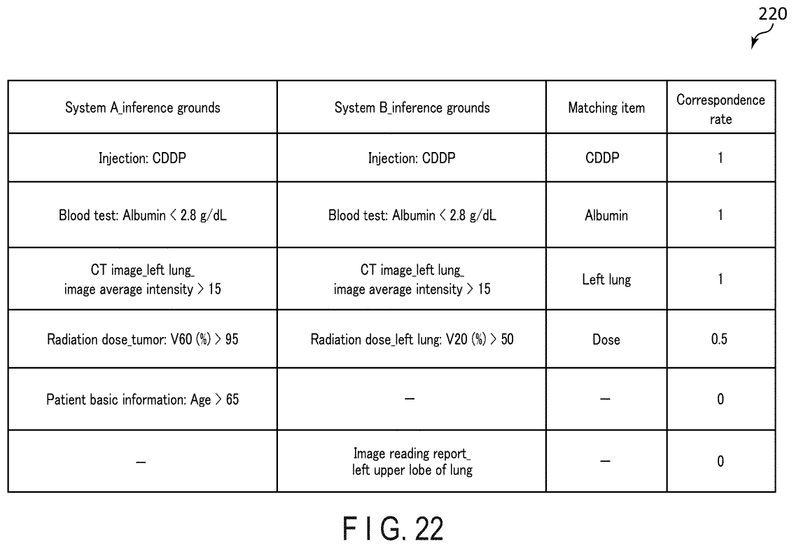

FIG. 22 is a table showing a combination of the matching item and the correspondence rate in the inference grounds of system A and system B according to the second embodiment. In a table 220 shown in FIG. 22, the system A_inference grounds, the system B_inference grounds, the matching item, and the correspondence rate are associated with each other. In FIG. 22, for example, the system A_inference grounds “Injection: CDDP”, the system B_inference grounds “Injection: CDDP”, the matching item “CDDP”, and the correspondence rate “I” are associated with each other.

(Step ST404)

After determining the correspondence rate, the processing circuitry 21 executes a matching rate calculation function 21g. By the matching rate calculation function 21g, the processing circuitry 21 calculates a matching rate of the inference grounds of the plurality of systems. Here, the matching rate is an indicator indicating the degree to which the inference grounds of the plurality of systems match. For example, the processing circuitry 21 calculates the matching rate based on the correspondence rate and a difference in importance level. The difference in importance level is calculated based on a difference in rank orders between the inference grounds of system A and the inference grounds of system B. The calculation method of the matching rate may be the same as that in the first embodiment.

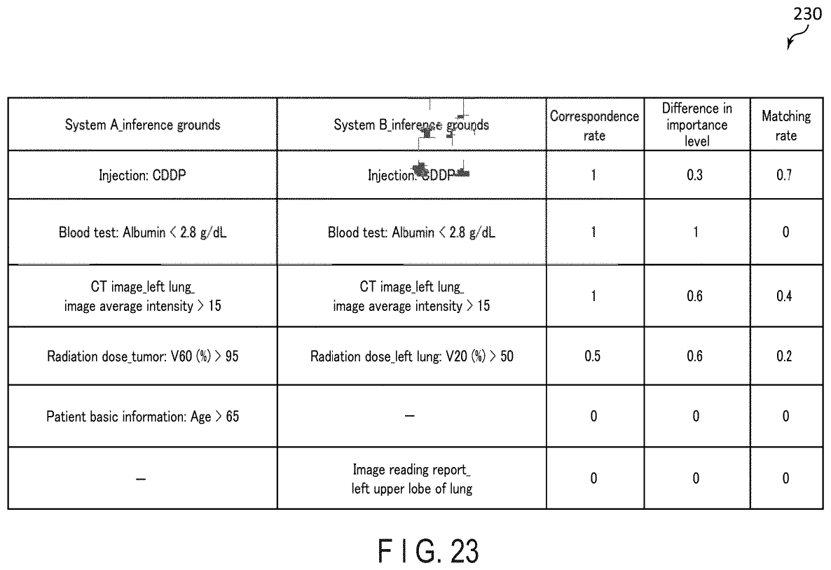

FIG. 23 shows a combination of the correspondence rate, the difference in importance level, and the matching rate in each item of the inference grounds of system A and system B according to the second embodiment. In a table 230 shown in FIG. 23, the system A_inference grounds, the system B_inference grounds, the correspondence rate, the difference in importance level, and the matching rate are associated with each other. In FIG. 23, for example, the system A_inference grounds “Injection: CDDP”, the system B_inference grounds “Injection: CDDP”, the correspondence rate “1”, the difference in importance level “0.3”, and the matching rate “0.7” are associated with each other.

(Step ST405)

After calculating the matching rate, by a display control function 21a, the processing circuitry 21 displays an inference result on a display 23. Here, the inference result includes, for example, a plurality of prediction probabilities relating to the inference target and a table indicating the inference grounds matching rate of system A and system B. Here, in the table indicating the inference grounds matching rate, for example, the inference information of system A, the inference information of system B, and the matching rate thereof are associated with each other.

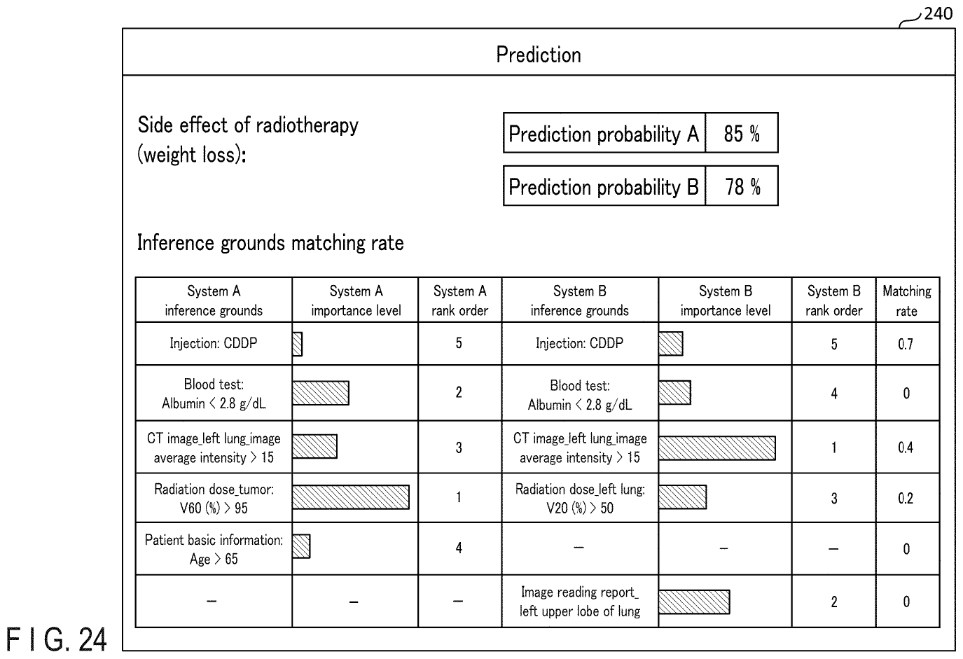

FIG. 24 shows a display example of the inference result according to the second embodiment. In an inference result 240 shown in FIG. 24, a plurality of prediction probabilities relating to the side effect of radiotherapy (weight loss), that is, the prediction probability of system A (prediction probability A) and the prediction probability of system B (prediction probability B) are shown. Furthermore, in the inference result 240, a table in which the system A_inference grounds, the system A_importance level, the system A_rank order, the system B_inference grounds, the system B_importance level, the system B_rank order, and the matching rate are associated with each other is shown.

Specifically, in the inference result 240, prediction probability A “85%” and prediction probability B “78%” are shown. Furthermore, as an example of the inference grounds matching rate, in the inference result 240, the system A_inference grounds “Injection: CDDP”, a graph corresponding to system A_importance level “0.05”, the system A_rank order “5”, the system B_inference grounds “Injection: CDDP”, a graph corresponding to system B_importance level “0.9”, the system B_rank order “5”, and the matching rate “0.7” are shown in an associated manner. Although the system A_importance level and the system B_importance level are expressed by bar graphs whose widths are each based on respective values, they may also be expressed by actual values.

(Step ST406)

After the inference result is displayed, by a determination function 21c, the processing circuitry 21 determines whether or not there are inference grounds with a matching rate equal to or higher than a threshold value in the inference result. In the case where there are inference grounds with a matching rate equal to or higher than the threshold value, the processing proceeds to step ST407, and, in the case where there are no inference grounds with a matching rate equal to or higher than the threshold value, the processing proceeds to step ST410.

(Step ST407)

After it is determined that there are inference grounds with a matching rate equal to or higher than the threshold value, by the display control function 21a, the processing circuitry 21 displays confirmation information for confirming items with a high matching rate and items with a high importance level on the display 23. The high matching rate indicates, for example, a value equal to or higher than the threshold value that is determined in step ST406.

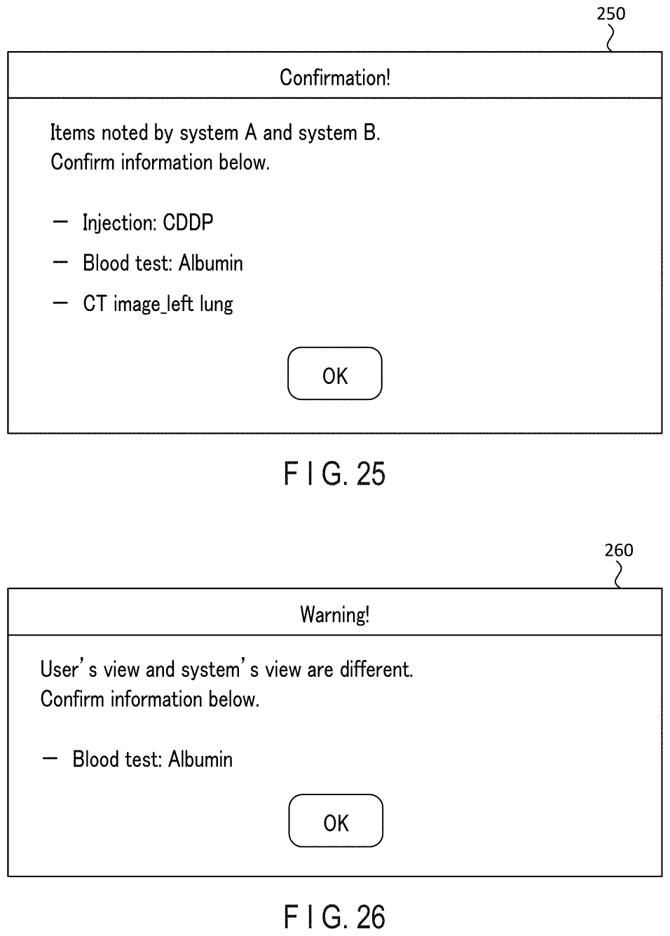

FIG. 25 shows a display example of the confirmation information according to the second embodiment. In confirmation information 250 shown in FIG. 25, for example, texts such as “Items noted by system A and system B. Confirm information below”, “Injection: CDDP”, “Blood test: Albumin”, and “CT image_left lung” are displayed.

For example, the item “Injection: CDDP” displayed in the confirmation information 250 is an item with the highest matching rate. Therefore, in order to encourage the user to confirm the item, the item is included in the confirmation information 250.

Furthermore, the item “Blood test: Albumin” displayed on the confirmation information 250 is an item with a high importance level in system A. Similarly, the item “CT image_left lung” displayed on the confirmation information 250 is an item with a high importance level in system B. Therefore, in order to encourage the user to confirm the items, these items are included in the confirmation information 250.

(Step ST408)

After the confirmation information is displayed, by the display control function 21a, the processing circuitry 21 displays a medical care screen on which an item with a high matching rate and an item with a high importance level are highlighted. Specifically, for example, the processing circuitry 21 displays an individual medical care information region corresponding to an item with a high matching rate and a high importance level on the medical care screen in a highlighted manner.

(Step ST409)

After displaying the medical care screen on which the item with a high matching rate and the item with a high importance level are highlighted, by the determination function 21c, the processing circuitry 21 determines whether or not the user has confirmed the highlighted items. In the case where the user has not confirmed the highlighted items (that is, in the case where the highlighted items remain), the processing is suspended until the user's confirmation is completed. In the case where the user has confirmed the highlighted items, the medical care decision support processing is ended.

(Step ST410)

After it is determined that there are no inference grounds with a matching rate equal to or higher than the threshold value, by the display control function 21a, the processing circuitry 21 displays confirmation information for confirming an item with a high importance level among the inferences of system A and system B on the display 23.

(Step ST411)

After the confirmation information is displayed, by the display control function 21a, the processing circuitry 21 displays a medical care screen on which the item with a high importance level is highlighted. Specifically, for example, the processing circuitry 21 displays an individual medical care information region corresponding to the item with the high importance level on the medical care screen in a highlighted manner.

(Step ST411)

After displaying the medical care screen on which the item with the high importance level is highlighted, by the determination function 21c, the processing circuitry 21 determines whether or not the user has confirmed the highlighted item. In the case where the user has not confirmed the highlighted item (that is, in the case where the highlighted item remains), the processing is suspended until the user's confirmation is completed. In the case where the user has confirmed the highlighted item, the medical care decision support processing is ended.

As explained above, the medical care support apparatus according to the second embodiment predicts the probability at which the event relating to the patient may occur by the first means, acquires the first grounds information constituting the grounds for the first means to arrive at the prediction, predicts the probability at which the event may occur by the second means which is different from the first means, acquires the second grounds information constituting the grounds for the second means to arrive at the prediction, calculates, based on the first grounds information and the second grounds information, a plurality of matching rates indicating the degree to which a plurality of items included in the first grounds information and a plurality of items included in the second grounds information match, and displays the inference result including a plurality of matching rates. In the second embodiment, the first means and the second means are, for example, algorithms that are different from each other. Accordingly, the medical care support apparatus is capable of calculating and displaying the matching rate after acquiring the inference grounds of a plurality of systems.

### Application Example of Second Embodiment

In an application example of the second embodiment, a case in which warning information is displayed based on the user inference information with respect to inference results provided by a plurality of systems in the second embodiment will be explained. In other words, in the present application example, a matching rate between the user inference grounds and the inference grounds of a plurality of systems is calculated.

By the matching rate calculation function 21g, the processing circuitry 21 calculates the matching rate between the user inference grounds and the inference grounds of system A. In a similar manner, the processing circuitry 21 calculates the matching rate between the user inference grounds and the inference grounds of system B. By the determination function 21c, the processing circuitry 21 determines whether or not there are inference grounds with a matching rate lower than a threshold value. After it is determined that there are inference grounds with a matching rate lower than the threshold value, by the display control function 21a, the processing circuitry 21 displays warning information indicating that there is a difference in the inferences between the user and the plurality of systems on the display 23.

FIG. 26 shows a display example of the warning information according to the application example of the second embodiment. In warning information 260 shown in FIG. 26, for example, texts such as “user's view and a plurality of system's view are different. Confirm information below.” and “Blood test: Albumin” are displayed.

“Blood test: Albumin” displayed on the warning information 260 is, for example, an item that was not presented as the inference grounds by the user. Therefore, since there is a possibility that the user may have overlooked the item, the item is included in the warning information 260.

In the manner explained above, the medical care support apparatus according to the application example of the second embodiment can prevent the user from overlooking the inference.

### Third Embodiment

In the second embodiment, the matter of displaying the inference result obtained by comparing the inference grounds between a plurality of systems has been explained. On the other hand, in a third embodiment, a matter of displaying an inference result obtained by comparing inference grounds between a plurality of users will be explained. Hereinafter, as a plurality of users, for example, two users, such as user A and user B, are assumed.

By an inference information acquisition function 21d, processing circuitry 21 acquires inference information of a plurality of users (user A inference information and user B inference information). By a correspondence rate determination function 21f, the processing circuitry 21 determines a correspondence rate between user A inference grounds and user B inference grounds. By a matching rate calculation function 21g, the processing circuitry 21 calculates a matching rate between the user A inference grounds and the user B inference grounds. By a display control function 21a, the processing circuitry 21 displays the inference result on a display 23. For example, the inference result includes a table indicating an inference grounds matching rate of user A and user B. In the table indicating the inference grounds matching rate, for example, the user A inference information, the user B inference information, and the matching rate thereof are associated with each other.

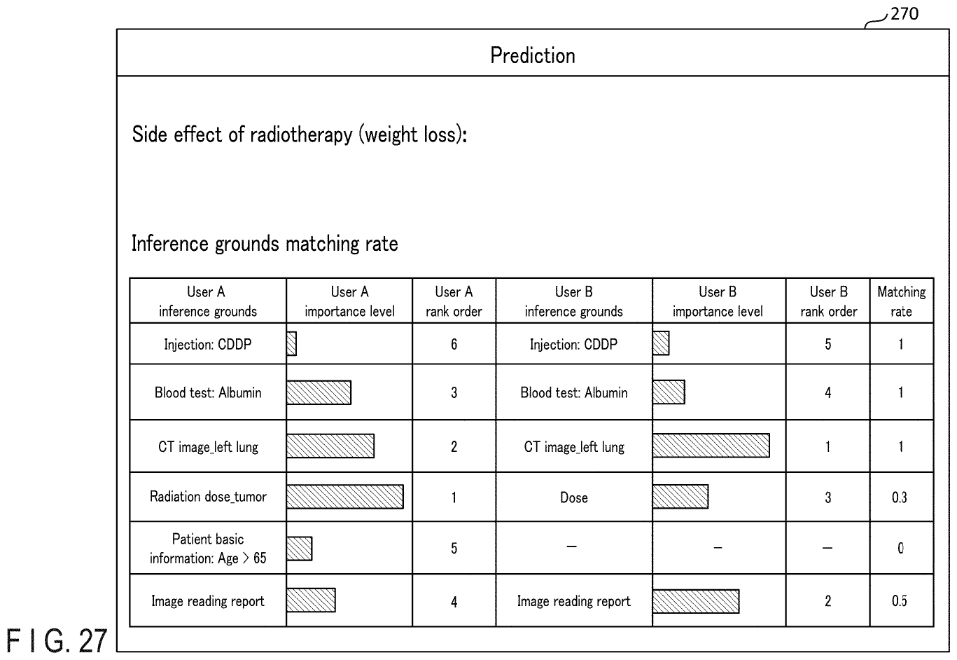

FIG. 27 shows a display example of the inference result according to the third embodiment. In inference result 270 shown in FIG. 27, a table in which user A_inference grounds, a user A_importance level, a user A_rank order, user B_inference grounds, a user B_importance level, a user B_rank order, and the matching rate are associated with each other is shown.

Specifically, in the inference result 270, as an example of the inference grounds matching rate, the user A_inference grounds “Patient basic information age >65”, a graph corresponding to the user A_importance level, the user A_rank order “5”, the user B_inference grounds “-” (i.e., no item), the user B_importance level “-”, the user B_rank order “-”, and the matching rate “0” are shown in an associated manner. Although the user A_importance level and the user B_importance level are expressed by bar graphs whose widths are each based on respective values, they may also be expressed by actual values. Furthermore, since the inference result 270 does not involve an inference by a system, a prediction probability is not shown.

By a determination function 21c, the processing circuitry 21 determines whether or not there are inference grounds with a matching rate lower than a threshold value. After it is determined that there are inference grounds with a matching rate lower than the threshold value, by the display control function 21a, the processing circuitry 21 displays warning information indicating that there are differences among the inferences of the plurality of users on the display 23.

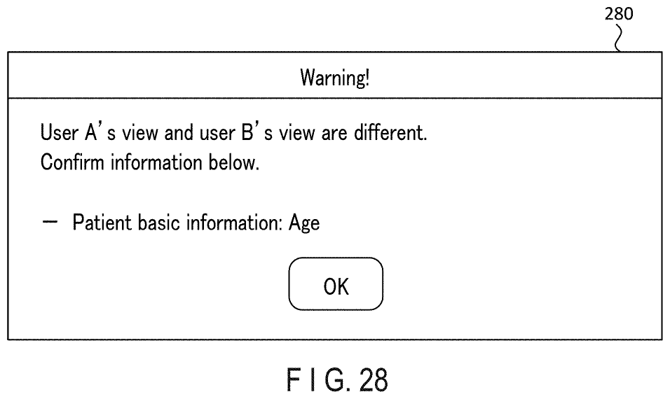

FIG. 28 shows a display example of the warning information according to the third embodiment. In warning information 280 shown in FIG. 28, for example, texts such as “user A's view and user B's view are different. Confirm information below.” and “Patient basic information: Age” are displayed.

“Patient basic information: Age” displayed on the warning information 280 is, for example, an item that was not presented as the inference grounds by user B. Therefore, since there is a possibility that the user may have overlooked the item, the item is included in the warning information 280.

In the manner explained above, the medical care support apparatus according to the third embodiment can compare inferences between a plurality of users.

According to at least one of the above-explained embodiments, it is possible to improve the accuracy of the decision.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical care support apparatus comprising processing circuitry configured to:
acquire first grounds information including grounds for a first means to arrive at a prediction of a probability at which an event relating to a patient may occur;
acquire second grounds information including grounds for a second means to arrive at a prediction relating to the event, the second means being different from the first means;
calculate, based on the first grounds information and the second grounds information, a plurality of matching rates indicating a degree to which a plurality of items included in the first grounds information and a plurality of items included in the second grounds information match; and
display an inference result including the plurality of matching rates.

2. The medical care support apparatus according to claim 1, wherein the processing circuitry is further configured to display the inference result in which a plurality of items included in the first grounds information, a plurality of items included in the second grounds information, and the plurality of matching rates are associated with each other.

3. The medical care support apparatus according to claim 1, wherein the processing circuitry is further configured to calculate the plurality of matching rates based on a plurality of first importance levels associated with each of a plurality of items included in the first grounds information and a plurality of second importance levels associated with each of a plurality of items included in the second grounds information.

4. The medical care support apparatus according to claim 3, wherein the processing circuitry is further configured to:
determine a plurality of correspondence rates indicating a corresponding relationship between a plurality of items included in the first grounds information and a plurality of items included in the second grounds information, respectively; and
calculate the plurality of matching rates based on the plurality of first importance levels, the plurality of second importance levels, and the plurality of correspondence rates.

5. The medical care support apparatus according to claim 4, wherein the processing circuitry is further configured to calculate the matching rates based on the correspondence rates and a difference in importance levels, the difference in importance levels being calculated for each item based on a difference between the first importance levels and the second importance levels.

6. The medical care support apparatus according to claim 4, wherein the processing circuitry is further configured to calculate the matching rates based on the correspondence rates and a difference in importance levels, the difference in importance levels being calculated for each item based on a difference in rank orders between a first rank order corresponding to the first importance levels and a second rank order corresponding to the second importance levels.

7. The medical care support apparatus according to claim 5, wherein the processing circuitry is further configured to calculate the matching rates for each item based on a ratio of between the correspondence rates and the difference in importance levels.

8. The medical care support apparatus according to claim 5, wherein the difference in importance levels is normalized in a predetermined numerical value range, and wherein the processing circuitry is further configured to calculate the matching rates for each item by multiplying the correspondence rate by a value obtained by subtracting the difference in importance levels from a maximum value of the predetermined numerical value range.

9. The medical care support apparatus according to claim 1, wherein the processing circuitry is further configured to:
determine whether or not the matching rates are lower than a threshold value; and
display, in a case where the matching rates are lower than the threshold value, information indicating that there is a difference between the first grounds information and the second grounds information, and display, in a case where the matching rates are equal to or higher than the threshold value, information indicating that the first grounds information and the second grounds information match.

10. The medical care support apparatus according to claim 9, wherein the processing circuitry is further configured to display, in a case where the matching rates are lower than the threshold value, an item relating to at least one of the first grounds information or the second grounds information in a highlighted manner on a medical care screen displaying medical care information of the patient.

11. The medical care support apparatus according to claim 1, wherein the processing circuitry is further configured to acquire the second grounds information by an input made by a user.

12. The medical care support apparatus according to claim 1, wherein the second means uses an algorithm different from that of the first means, and wherein the processing circuitry is further configured to acquire the second grounds information in which a probability at which the event may occur is predicted by the second means.
